(19) United States
(12) Patent Application Publication
(10) Pub. No.: US 2005/0020582 A1
(43) Pub. Date: Jan. 27, 2005

(54) Optically active compounds clearing malformed proteins

(76) Inventors: Stanley B Prusiner, San Francisco, CA (US); Carsten Korth, Dusseldorf (DE); Barnaby C. H. May, San Francisco, CA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/478,560

(22) PCT Filed: May 23, 2002
(86) PCT No.: PCT/US02/16349

Related U.S. Application Data
(60) Provisional application No. 60/293,771, filed on May 25, 2001.
(60) Provisional application No. 60/293,705, filed on May 25, 2001.
(60) Provisional application No. 60/301,345, filed on Jun. 26, 2001.
(60) Provisional application No. 60/329,602, filed on Oct. 15, 2001.

(51) Int. Cl. A61K 31/5415; A61K 31/473

(52) U.S. Cl. 514/225.8; 514/297

(57) ABSTRACT

The invention is drawn to compositions and methods for inhibiting and treating malformed forms of proteins causing neurodegenerative disease, such as protease resistant prion proteins (PrP^{Sc}) and those associated with transmissible spongiform encephalopathies (TSEs). The compounds disclosed herein can be present as racemic mixtures or as compositions consisting essentially only of the optically active isomer in a higher percentage amount e.g. 60% or more, 70% or more, 80% or more, 90% or more or 100% of the optically active isomer and specifically the dextrorotary isomer of quinacrine.

### FIELD OF THE INVENTION

The invention relates to compounds which act as inhibitors of malformed proteins, such as, for example, protease resistant prion proteins (PrP^{Sc}) and those associated with transmissible spongiform encephalopathies (TSEs), and methods of their use.

### BACKGROUND OF THE INVENTION

Diseases associated with pathogenic forms of proteins, e.g., PrP^{Sc}, have received increased public attention since the outbreak of bovine spongiform encephalopathy (BSE) in Great Britain in 1984 and the subsequent discovery of its transmissibility to humans causing a variant of Creutzfeldt-Jakob disease (CJD). Many other diseases are known to be associated with pathogenic proteins, such as systemic lupus erythematosus, arthritis, multiple sclerosis, etc. See, Wojtowicz, S., Med Hypotheses 40(1):48-54 (1993) and Weller, R. O., J. Neuropathol. Exp. Neurol. 57(10):885-894 (1998).

As with many other neurodegenerative diseases, no cure is known at this time. As such, a need exists for the discovery of therapeutic compounds and methods of their use so that these neurodegenerative diseases can be treated. See, Prusiner, S. B., N Engl. J. Med. 344(20):1548-1551 (May 17, 2001).

PrP^{Sc} is an infectious protein that causes central nervous system spongiform encephalopathies in humans and animals. It has been shown that a scrapie isoform of the prion protein (PrP^{Sc}) is necessary for both the transmission and pathogenesis of the transmissible neurodegenerative diseases of animals and humans. See, Prusiner, S. B., Science 252:1515-1522 (1991). The most common prion diseases of animals are scrapie of sheep and goats and bovine spongiform encephalopathy (BSE) of cattle. See, Wilesmith and Wells, Microbiol. Immunol. 172:21-38 (1991).

Examples of human neurodegenerative prion-related diseases include: (1) kuru, (2) Creutzfeldt-Jakob Disease (CJD), (3) Gerstnann-Strassler-Scheinker Disease (GSS), and (4) fatal familial insomnia (FFI).

These diseases are characterized by the formation and accumulation of an abnormal protease resistant isoform (PrP^{Sc}) of a normal protease-sensitive endogenous prion protein (PrP^{C}). The protease-resistant isoform accumulates in the CNS and other tissues.

As such, it would be desirable to identify compounds and methods for treating neurodegenerative diseases wherein the formation or accumulation of pathogenic forms of proteins, such as, protease resistant PrP^{Sc} proteins, can be circumvented by suitable therapeutic intervention with pharmaceuticals.

It would also be desirable to identify methods for screening test compounds for the ability to inhibit formation or accumulation of these pathogenic proteins.

Based on the foregoing, it is clear that a need exists for identification of compounds that may treat neurodegenerative diseases, and methods of their use. The present invention fulfills these and other needs in the art.

### SUMMARY OF THE INVENTION

The invention comprises a method of clearing pathogenic forms of proteins from cells, by contacting the cells with a therapeutically effective amount of a compound having a multicyclic scaffold and a side chain, wherein the scaffold has at least two, preferably, three cyclic moieties. With regard to all structures shown here and compounds named all stereoisomers are included when such exist including optical isomers, racemic mixtures and pure enantramers.

The method of the present invention comprises administering a compound having can have the following general structural formula I: 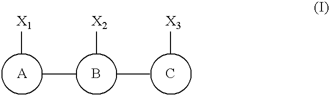
wherein “A,” “B” and “C” are each independently a cyclic moiety comprised of atoms selected from the group consisting of carbon, nitrogen and sulfur, any of which cyclic moieties may be substituted at any position, and each of X₁, X₂ and X₃ is independently a hydrocarbyl as defined here and is preferably a nitrogen containing non-cyclic hydrocarbyl moiety covalently bound to B. Preferably A, B and C are six-membered rings and A and C are comprised only of carbon and hydrogen and B preferably comprises carbons and a nitrogen or sulfur atom.

The compound which is administered can also have the following general structural formula II: 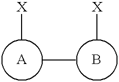
wherein “A” and “B” are each independently a cyclic moiety comprised of atoms selected from the group consisting of carbon, nitrogen and sulfur, any of which cyclic moieties may be substituted at any position and “X” is a hydrocarbyl as defined here and is preferably a nitrogen containing non-cyclic hydrocarbyl moiety covalently bound to A. Preferably A and B are five-membered rings comprised of carbon and hydrogen and B preferably comprises a nitrogen and/or sulfur.

Preferably, the cyclic moieties of the compound are fused rings, more preferred is that the moieties be six-membered rings. The rings can be comprised of unsaturated carbons and it is preferred that moiety B is heterocyclic with the heteroatoms comprising nitrogen, sulfur and oxygen most preferably a single nitrogen or sulfur atom.

Examples of the cyclic moieties of the compound comprise the following structures:

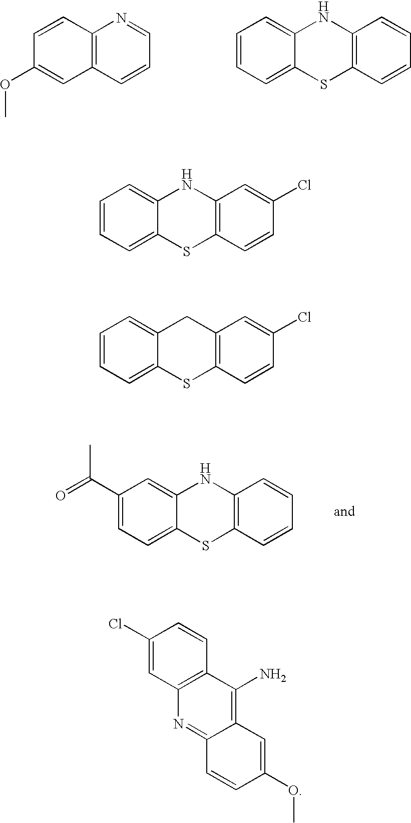

It is preferred that a side chain, X, is bound to B via a carbon or a nitrogen atom of B and X is alkyl (comprising a hetero atom which is N, S or O) comprising 1 to 8 carbon atoms having the general formula CₙH₂ₙ₊₂ preferably having a hydrocarbon chain of at least about C₃H₈ more preferably C₄H₁₀.

More preferably, X further comprises an amine substituent, such as, for example, a dimethylamino group. It is preferred that the amine substituent further comprises at least two substituents, preferably, two methyl groups. As such, several examples of amine substituents of X comprise a methylamine, an ethylamine, 1-propylamine and 1-butylamine. The amine substituent may be bifurcated or unbifurcated, as in for example, a methyl bifurcation.

Preferred structural formulae of side chain X comprise:

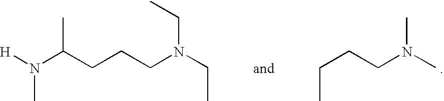

Compounds administered in the method of the present invention comprise phenothiazine and acridine derivatives, e.g., phenothiazine, promazine, chlorpromazine, acepromazine, quinacrine and pamaquine. The most preferred compounds are quinacrine and chlorpromazine.

An unexpected finding is that compounds with a multicyclic scaffold and a side chain, especially a tricyclic scaffold with a side chain substituted middle ring, are effective inhibitors of the formation of protease resistant prions, PrP^{Sc}, and as such, these compounds can be used to treat pathogenic protein diseases.

The invention further comprises a method of treating a disease characterized by pathogenic protein formation, by administering a pharmaceutically effective amount of a combination of quinacrine and chlorpromazine to a subject. The use of these compounds in combination has a synergistic effect, and is not simply additive. This finding was unexpected.

The invention also includes a pharmaceutical composition comprising a compound of the above described general formulae (I and II), or a variant or mimetic thereof, and a pharmaceutically acceptable carrier or diluent.

Compounds of the present invention can be administered in a pharmacological composition with pharmaceutically acceptable carriers, fillers or excipients. Such a composition can also include a lipophilic solvent or carrier, such as DMZ, an organic solvent, phosphatidyl choline or cholesterol.

Treatment is by administering a therapeutically effective amount of the pharmaceutical composition, either singly or in combination, to a mammal that has been exposed to and/or is in danger of being exposed to the transmissible agent, such as PrP^{Sc}, or which is exhibiting signs, symptoms or laboratory evidence of a TSE. If the mammal is merely suspected of having been exposed to a TSE, the treatment is a prophylactic method of preventing the progression of the disease. In a situation where the mammal is already believed to be exhibiting signs or symptoms of the disease, the treatment is also a method of improving the neurological or other biological condition of the animal.

The invention also includes in vitro methods for the inhibition of the conversion of prPc to PrP^{Sc}, and a method of screening for such compounds and including variants, analogs and mimetics of the inhibitory compounds that inhibit the conversion reaction. The screening method includes contacting PrP^{C} with PrP^{Sc} or analogs, derivatives or mimetics thereof in the presence of a test compound, and determining whether the test compound, analog, derivative or mimetic inhibits conversion of PrP^{C} to PrP^{Sc}. Specific preferred embodiments of the present invention will become more evident from the following detailed description.

An aspect of the present invention includes methods of administering to a mammal, one or more compounds from a class of compounds, having a general structural formula I or II or compounds from both of these classes.

In another aspect of the invention, a mammal in need of this treatment is identified, and a pharmacologically effective amount of a compound is administered to the mammal in an amount sufficient to interfere with PrP^{Sc} formation or accumulation in cells.

The present invention includes still another aspect comprising treating a mammal, such as a human, having a condition associated with PrP^{Sc}. In this aspect, such a mammal is identified and treated with a compound, either singly or in combination, of the present invention in a manner, such as that described above in the foregoing aspects of the invention.

A method of the invention comprises administering a compound to a cell population, wherein said cell population is exposed to an amount of stimulus capable of inducing the formation of protease resistant prion proteins in the cell population, and determining whether the presence of said compound inhibits PrP^{Sc} formation or accumulation and, typically, also produces a detectable reduction in the amount and/or rate of PrP^{Sc} in the cell population; if said agent produces PrP^{Sc} inhibition in cells and/or inhibits conversion of PrP^{C} to PrP^{Sc}, the compound is thereby identified as a therapeutic compound. Preferably, the method is used to demonstrate that the compound inhibits PrP^{Sc} formation or accumulation and also inhibits neuronal degenerative diseases (e.g., transmissible spongiforn encephalopathies).

In a variation of the method, the agent is initially selected from a bank (or library) of compounds on the basis of the agent's chemical structure for inhibiting PrP^{Sc} in vitro; an agent which is thus initially selected is administered to a cell population, wherein said cell population is exposed to an amount of stimulus capable of inducing protease resistant prion proteins in the cell population, and the capacity of said compound to produce a detectable reduction in the amount and/or rate of PrP^{Sc} in the cell population is determined, with compounds capable of reducing PrP^{Sc} being thereby identified as active agents. In this variation, the capacity of the agent to selectively or specifically inhibit PrP^{Sc} in a cultured cell population can optionally be determined.

In a further aspect, the invention also provides a method for identifying an active agent which significantly inhibits neuronal degeneration in a transgenic animal model; such active agents can be sold commercially to control the disease in animals for any purpose desired by an end-user of such animals, and can serve as candidate pharmaceuticals for therapy of neurodegenerative disease, among other uses. The method comprises initially selecting an PrP^{Sc}-inhibiting agent from a bank (or library) of compounds on the basis of: (1) the agent's capacity, selectivity, or specificity for inhibiting PrP^{Sc} in vitro, such as by its ability to inhibit PrP^{Sc} formation or accumulation in an in vitro assay and/or (2) the capacity of the agent to selectively inhibit PrP^{Sc} in a cultured cell population; and administering the selected agent to a transgenic animal capable of developing detectable pathology characteristic of a PrP^{Sc} related neurodegenerative disease, and determining whether administration of the selected agent inhibits or retards development of said detectable pathology as compared to a substantially identical identifying control transgenic animal which lacks the agent; an agent which retards or inhibits development of neuropathology is thereby identified as an active agent.

In a further aspect, the invention provides a method for reducing or retarding neurodegeneration in a cell population comprising neurons which have been exposed to an amount of a stimulus sufficient to produce partially protease resistant proteins resulting in neurodegeneration; said method comprising administering an efficacious dose of a PrP^{Sc} inhibitor predetermined to retard or inhibit neuronal degeneration. In one embodiment, the cell population may reside in the central nervous system of a mammal and the PrP^{Sc} inhibitor is administered in vivo. The invention also provides the use of a PrP^{Sc} inhibitor to treat neurodegenerative disease pathology in a mammal.

In a still further aspect, the invention provides a method for retarding or inhibiting neurodegeneration in a cell population comprising neurons exposed to an amount of stimulus sufficient to produce protease resistant protein related neurodegeneration; said method comprising administering to the cell population an efficacious dose of a compound capable of inhibiting expression of PrP^{Sc}. In one embodiment, the cell population may reside in the central nervous system of a mammal and the PrP^{Sc} inhibitor is administered in vivo.

Also provided by the invention is a method for inhibition of neuronal cell death in a cell population. The method comprises delivering an effective dosage of a PrP^{Sc} inhibitor to a cell population that is exposed to a protease resistant prion protein stimulus.

Another aspect of the invention is that a compound of the invention which is characterized by its ability to inhibit prion formation and allow for clearing of prions can be combined with a pharmaceutical product; in a particular a product derived from a human source such as organs, tissue, blood, and related blood derived products.

In another aspect of the invention is a method of treating tissue, organs, blood and blood derived products by combining such with a compound of the invention.

In another aspect of the invention is the combination of livestock feed with a compound of the invention, which is particularly useful in connection with livestock feed which includes meat, bone meal or any material derived from an animal that might be infected with prions.

In another aspect of the invention is a method of treating farm animals by administering to farm animals a compound of the invention which compound of the invention may be combined with livestock feed thereby preventing prion infections and/or treating prion diseases such as “mad cow” disease in animals consuming the treated livestock feed.

These and other aspects of the invention will be understood by those skilled in the art upon reading this disclosure.

### BRIEF DESCRIPTION OF THE FIGURES

FIG. 1 shows the structural formula of twelve different compounds each accompanied by an image of an immunoblot run with each compound showing the relative amounts of protease resistant PrP^{Sc} reduction observed in permanently scrapie-infected neuroblastoma cells (ScN2a) after a week of treatment with these concentrations (1, 5, and 10 μM) of each of the twelve compounds.

FIG. 2a shows images of three immunnoblots and a graph, which demonstrates a dose-response relationship between the amount of chlorpromazine and PrP^{Sc} inhibition.

FIG. 2b shows images of three immunoblots and a graph, which demonstrates that quinacrine has a ten-fold higher potency on PrP^{Sc} inhibition as compared to chlorpromazine.

FIG. 2c shows images of three immunoblots that measure the anti-PrP^{Sc} potency of methylene blue, a cytotoxic tricyclic compound.

FIG. 3 shows the structural formula of eleven different compounds along with an image of an immunoblot run with the compound showing the PrP^{Sc} inhibiting effects of these six structurally similar compounds.

FIG. 4 is an image of a gel showing the relative decrease of PrP^{Sc} in cells after six days following quinacrine treatment (left panel) and the relative amounts of PrP^{Sc} in cells three weeks after discontinuation of quinacrine treatment (right panel).

FIG. 5A is a western blot of protease K-digested ScN2a cell lysates incubated with (+)-quinacrine at different micromolar concentrations as shown at the top of each column.

FIG. 5B is a western blot of protease K-digested ScN2a cell lysates incubated with (−)-quinacrine at different micromolar concentrations as shown at the top of each column.

### DETAILED DESCRIPTION

Before the present formulations and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and” and “the” include plural referents unless the context clearly dictates otherwise. Thus, for example, reference to “a compound” includes a plurality of such compounds and reference to “the step” includes reference to one or more steps and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided might be different from the actual publication dates, which may need to be independently confirmed.

Definitions

The terms “treatment,” “treating,” “treat” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in an animal, particularly a human, and includes:
(a) preventing the disease or symptom from occurring in a subject which may be predisposed to the disease or symptom but has not yet been diagnosed as having it;
(b) inhibiting the disease of its symptom, i.e., arresting development of the disease or its symptoms; or
(c) relieving the disease symptom, i.e., causing regression of the disease or symptom.

The term “hydrocarbyl” includes hydrocarbon as well as substantially hydrocarbon groups. Substantially hydrocarbon describes groups which contain non-hydrocarbon substituents which do not alter the predominantly hydrocarbon nature of the group.

Examples of hydrocarbyl groups include the following:
(1) hydrocarbon substituents, that is, aliphatic (e.g., alkyl or alkenyl), substituents which may be a straight or branched chain containing from —H at a low end to 30 carbons at an upper end which carbons may be saturated or not with hydrogens;
(2) substituted hydrocarbon substituents, that is, those substituents containing non-hydrocarbon groups which, in the context of this invention, do not alter the prion inhibitor characteristics of the compound; those skilled in the art will be aware of such groups (e.g., halo, chloro, fluoro, hydroxy, alkoxy, mercapto, alkylmercapto, nitro, nitroso, sulfoxy); and
(3) hetero substituents, that is, substituents that, while having a predominantly hydrocarbon character within the context of this invention, contain an atom other than carbon present in a chain or branched chain otherwise composed of carbon atoms. Suitable hetero atoms will be apparent to those of ordinary skill in the art and include, for example, sulfur, oxygen, nitrogen, phosphorus and such substituents as, e.g., pyridyl, furyl, thienyl, imidazolyl, etc. In general, at least about 2, preferably no more than about 5, non-hydrocarbon substituents will be present for every ten-carbon atoms in the hydrocarbyl group. Some preferred hydrocarbyl groups are H, and substituted hydrocarbon substituents in category (2) above. Particularly preferred substituents for “X” include:
   —H, —NH₂, —CH₂CH₂CH₂N(CH₃)₂; and
   —NHCH(CH₃)(CH₂)₃N(C₂H₅)₂.

### Overview

A basis of the invention is the discovery that compounds with certain characteristics (e.g., compounds having a multicyclic scaffold and a side chain) have anti-PrP^{Sc} potency that is useful in the treatment of mammals infected with or exposed to prions. It is believed that certain chemical structures contribute an essential component to their anti-PrP^{Sc} activity. It is preferred that these anti-PrP^{Sc} compounds comprise phenothiazine and its derivatives, more preferably, chlorpromazine. It is also preferred that these compounds comprise acridine and its derivatives, more preferably, quinacrine. It is most preferred that quinacrine and chlorpromazine be administered in combination.

The preferred cyclic backbone of compounds used in the invention is:

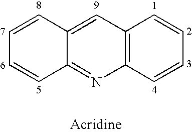

The numbering scheme shown above is used here to describe molecules used in the invention. Preferred compounds used in the invention have the side chain “X” attached at the “9” position. Preferred examples include the following: 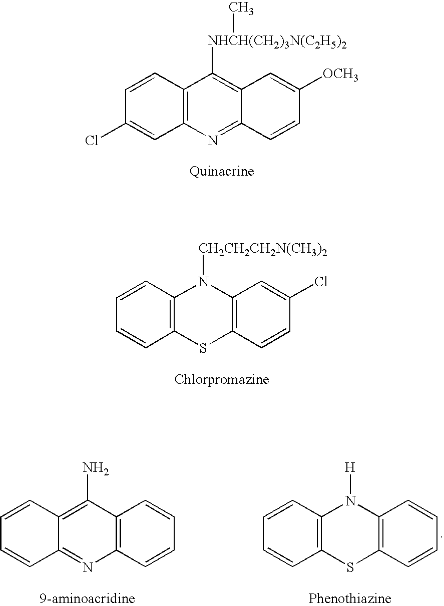
Dosing

One or more compounds of the invention can be administered by any desired route of administration, including injection, IV or IM, implanted pump, transdermal, intrapulmonary, intranasal, etc. However, oral administration is generally preferred and such can include a quick release or controlled release formulation. The mode of administration will generally affect dosing in that some modes are more efficient at delivery of active compound to the desired site than others.

A range of factors are known to affect dosing including the size, weight, sex, age and condition of the patient. Those skilled in the art will adjust dosing as needed, beginning with smaller doses and increasing gradually while monitoring side effects and the effect of the drug on the disease being treated.

With an oral formulation of a compound such as quinacrine, dosing is generally in an amount of about 100-10,000 mg/day/75 kg of body weight of the animal being treated. Thus, a human dose is about 100-10,000 mg/day, and larger animals are given larger doses in proportion to their weight. It should be noted that the efficacy of a compound on cells is some indication of the potency of the compound. However, some compounds cross the blood-brain barrier more efficiently than others and such is to be considered in dosing.

When quinacrine and chlorpromazine are given in combination, the total dosage is generally the same as for quinacrine alone. That is, 10-10,000 mg/day per 75 kg of body weight. The compounds used in the combination therapy may be applied serially, in any order, or both compounds may be administered at the same time. Preferentially, the combination of compounds is given at the same time to the subject. One of skill in the art would know how to manipulate the ratio of dosages to provide for an optimal result.

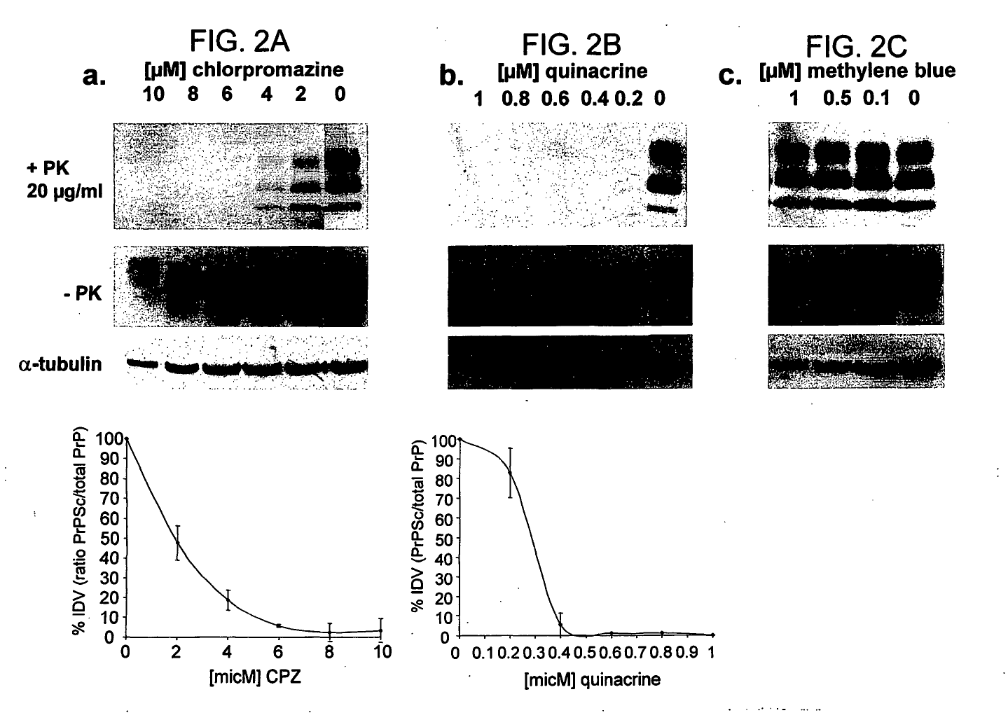

A dose-dependent relationship was established for increasing doses of inhibitors of PrP^{Sc} for quinacrine and chlorpromazine. PrP^{Sc} inhibition was observed to have a weak effect after only two days and a substantial effect after seven days of treatment of ScN2a cells (FIG. 2a). For example, when cells were treated with a 2 μM dose of chlorpromazine, the amount of PrP^{Sc} diminished by 50%. Western blot densitometry showed a linear decrease of PrP^{Sc} in a range of about 1 to 10 μM (FIG. 2a, lower panel). At a dose of 10 μM, chlorpromazine exhibited cytotoxic effects, as can be seen by a 50% decrease in cell population and a reduction in PrP^{Sc} in the cell lysate when the 10 μM dosage was compared with other dosage conditions (FIG. 2a).

To account for the possibility that cytotoxicity was responsible for the PrP^{Sc} inhibition, cells were tested with a known cytotoxic tricyclic compound, methylene blue. Because methylene blue did not exhibit any anti-PrP^{Sc} effects (FIG. 2c), a tricyclic ring and mid-ring side chain structure is demonstrated to play a critical role in the inhibition of PrP^{Sc}.

Phenothiazines are known antipsychotic compounds that have been proposed to be dopamine antagonists. To control for the possibility that the phenothiazines are inhibitors of PrP^{Sc} formation because of their dopamine antagonist properties, structurally unrelated high potency antipsychotic drugs, haloperidol and clozapine, were tested on the cell model. Because the haloperidol and clozapine did not inhibit PrP^{Sc} formation or accumulation in the range tested, the PrP^{Sc} inhibiting effect of phenothiazine and its derivatives is not believed to be mediated through their dopamine antagonistic property.

Quinacrine, an acridine derivative and a tricyclic compound with a side chain anchored to its middle ring, exhibited a tenfold higher potency on PrP^{Sc} as compared to a phenothiazine derivative, chlorpromazine. Quinacrine effectively inhibited PrP^{Sc} at a concentration of at least about 200 nM and completely inhibited PrP^{Sc} formation at a concentration of about 400 nM (FIG. 2b). Quinacrine did not exhibit cytotoxic effects on neuroblastoma cells at concentrations <5 μM and as such, quinacrine has a large therapeutic range (FIG. 2b). Quinacrine may also be administered in combination with chlorpromazine. Surprisingly, the compounds appear to have a synergistic effect in denaturing prions in cultured cells.

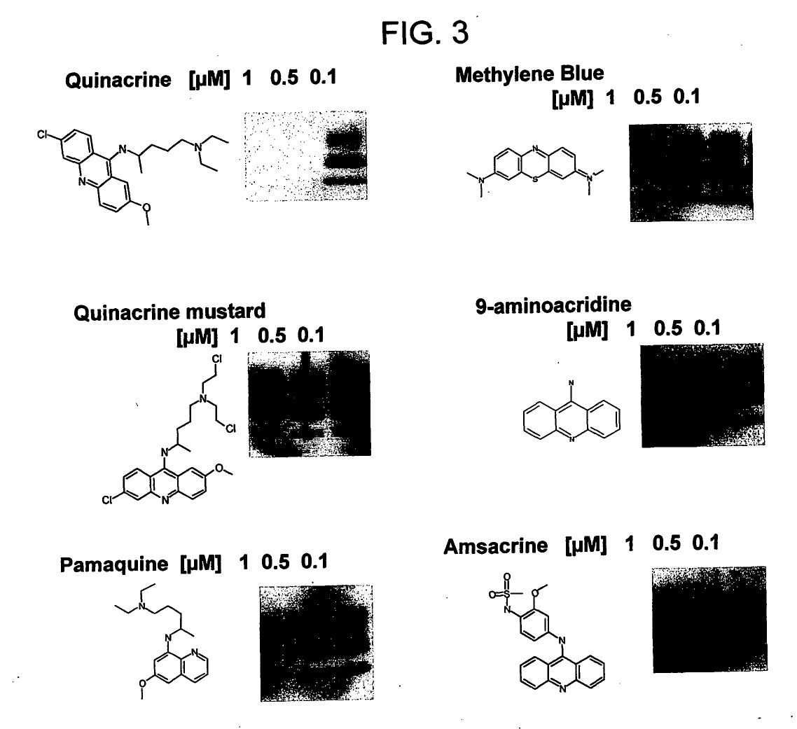

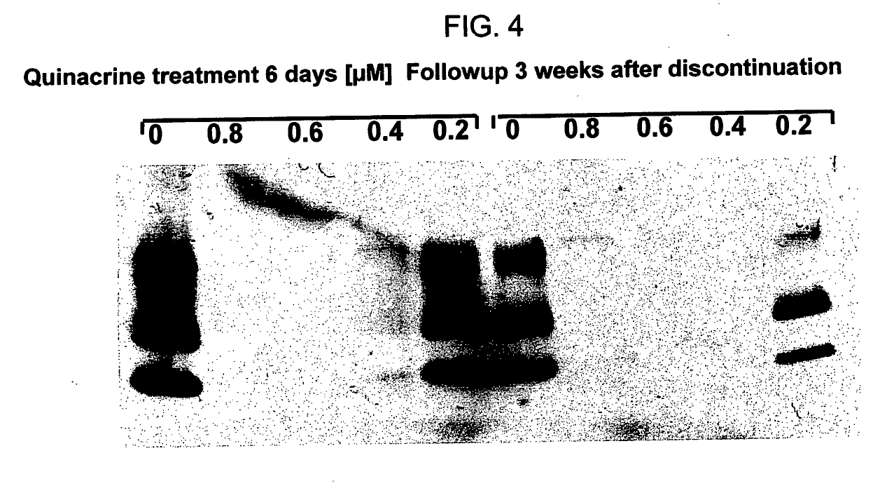

A comparison of anti-PrP^{Sc} activity among eleven compounds, e.g., quinacrine, quinacrine mustard, pamaquine, methylene blue, 9-aminoacridine and amsacrine is illustrated in FIG. 3. As such, the length and composition of the side chain is an important feature of the PrP^{Sc} inhibitory structure. Further, a quinacrine side chain anchored to pamaquine or chloroquine, a dicyclic structure, see FIG. 3, did not inhibit PrP^{Sc} at the same potency as quinacrine, thus bolstering the importance of the tricyclic structural backbone for anti-PrP^{Sc} potency.

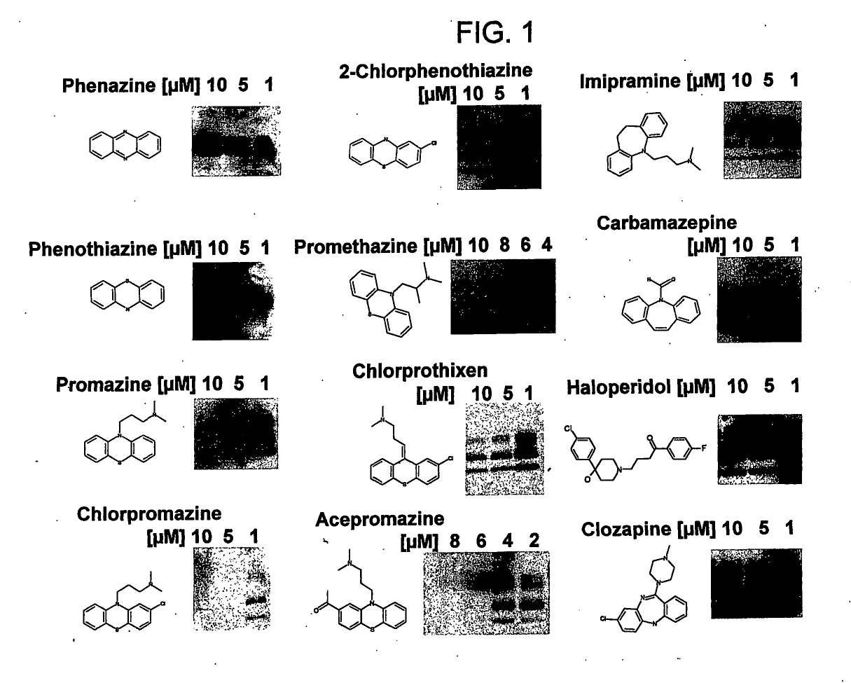

As can be seen in the structural formulae (FIG. 1), compounds having a tricyclic ring structure and a side chain attached to the middle ring were more potent inhibitors of PrP^{Sc} formation as compared to compounds which lacked such structures. Following experiments with test compounds, phenothiazine derivatives and structurally related compounds lead to a decrease in the amount of protease resistant PrP^{Sc} after a week of treatment (FIG. 1). Preferred concentrations of the compounds are from about 1 to 10 μM, preferably, 2 to 8 μM in order to maximize PrP^{Sc} reduction and minimize cytotoxicity.

This new class of protease resistant prion protein inhibitors is structurally similar to compounds capable of inhibiting PrP^{Sc} formation and accumulation in cells such as that which occurs in human neurodegenerative diseases. Agents that inhibit PrP^{Sc} formation or accumulation can inhibit such neuropathological changes and thus can be used to treat associated neural degenerative effects, as well as other diseased characterized by an abnormal protein formation.

PrP^{Sc} related diseases may be inhibited by compounds having a tricyclic ring structure and a side chain anchored to its middle ring. As such, the present invention describes a method of inhibiting the formation or accumulation of PrP^{Sc} proteins in vitro and in vivo by administering to a subject a pharmacologically effective amount of an anti-PrP^{Sc} compound in an amount sufficient to interfere with PrP^{Sc} formation and accumulation in cells. It is preferred that a compound or compounds of the present invention, be administered in an amount such that it results in a concentration with the treated cells in a range of about 1 to 10 μM, preferably about 2 to 8 μM.

The transmissible dementias or spongioform encephalopathies such as Creutzfeldt-Jakob disease, Gerstmann-Straussler syndrome, kuru and a variety of other diseases such as scrapie, are all characterized by abnormal accumulations of PrP^{Sc}. As such, a mammal suffering from such accumulation can be effectively treated by administration of a formulation of the invention. For instance, chlorpromazine (or an effective derivative of phenothiazine) can be given to a mammal in need by oral or parenteral administration. Preferred administration methods include intravenous injection, transdermal administration, intraperitoneal injection, subcutaneous injection, intramuscular injection, intrasternal injection, intrathecal injection, intranasal or direct infusion techniques. Osmotic pumps, which administer a formulation of the invention directly to cells to be treated (e.g., brain tissue) can be used.

Optically Active Isomers

Many of the compounds disclosed herein are present as racemic mixtures i.e. 50/50 mixtures of both the dextrorotary (D) also (+) and laevototary (L) also (−) optically active components. The present invention includes not only the use of the racemic mixture but either of the optically active isomers by themselves or present in larger concentrations that a 50% concentration within the racemic mixture. In a preferred example the (D) isomer is isolated in a 100% concentration and used to clear malformed proteins such as prions from cells. The optically active isomer can, of course, be added to animal feed as described herein or used to treat animals or humans as described herein. The (D) isomer may be present in an amount of 100% of the active compound, 90%, 80%, 70% or 60% with the remainder of the compound being the (L) isomer.

Prophylactic Uses

As indicated here, the term “treating” means preventing, inhibiting or relieving the disease or symptom thereof. However, there are specific situations wherein a clear prophylactic treatment is indicated, i.e., the aspect of treating that involves preventing the disease. Specifically, there is an identifiable portion of the population that has inherited diseases related to prions such as CJD. Family members who have or might have an inherited trait for such disease can take a composition of the invention in order to prevent the development of the disease and/or symptoms. A second class of individuals are those who have been exposed to prions by ingesting infected food such as beef products, which were derived from cattle with BSE. This would include individuals who have spent significant amounts of time in Europe and/or other areas where the cattle were likely to have been infected with BSE. A third group of people are people who have been treated with human growth hormone derived from a human cadaver. Such individuals are at risk for the development of such diseases and would be treated with a composition of the invention as described herein. A fourth group of individuals are those that have been subjected to surgery and exposed their nerve tissue to surgical instruments, which may have been already infected with prions. Further, the individual may be an individual who has had a dura mater transplant from an individual who may have been infected with a disease such as CJD.

### Diseases and Their Malformed Proteins

The invention also includes a method of treating pathogenic protein diseases, like protease resistant prion related diseases. Diseases that may be treated by the claimed methods include, but are not limited to TSE, CJD, Alzheimer's disease, Parkinson's disease, amyotrophic lateral sclerosis, multiple sclerosis, autism, schizophrenia, bipolar disorders, fronto-temporal dementia, Pick's disease, progressive supranuclear palsy, diffuse Lewy body disease, systemic lupus erythematosus, rheumatoid arthritis, Huntington's disease, spinocerebellar ataxias, diabetes mellitus, Types I and II, Crohn's disease, ulcerative colitis, systemic amyloidosis, primary amyloidosis, polyneuropathy and AIDS.

Much of the disclosure and the specific examples provided herein relate to the use of compounds and combinations of compounds in the clearance of PrP^{Sc} from a sample. However, as indicated above, the compounds and methods of the invention can be applied to obtain the clearance of or prevent the formation of malformed proteins for any protein which assumes two different conformational shapes, one of which is associated with the disease. The following is a non-limiting list of diseases with associated insoluble proteins which assume two or more different conformations.

| Disease | Insoluble Proteins |
|---|---|
| Alzheimer's Disease | APP, Aβ peptide, α1-antichymotrypsin, |
| | tan, non-Aβ component |
| Prion diseases, Creutzfeld Jakob | PrP^{Sc} |
| disease, scrapie and bovine | |
| spongeform Encephalopathy | |
| ALS | SOD and neurofilament |
| Pick's disease | Pick body |
| Parkinson's disease | Lewy body |
| Diabetes Type 1 | Amylin |
| Multiple myeloma—plasma cell | IgGL-chain |
| dyscrasias | |
| Familial amyloidotic | Transthyretin |
| polyneuropathy | |
| Medullary carcinoma of thyroid | Procalcitonin |
| Chronic renal failure | β₂—microglobulin |
| Congestive heart failure | Atrial natriuretic factor |
| Senile cardiac and systemic | Transthyretin |
| amyloidosis | |
| Chronic inflammation | Serum amyloid A |
| Atherosclerosis | ApoA1 |
| Familial amyloidosis | Gelsolin |

It should be noted that the insoluble proteins listed above each include a number of variants or mutations which result in different strains which are all encompassed by the present. Known pathogenic mutations and polymorphisms in the PrP gene related to prion diseases are given below and the sequences of human, sheep and bovine are given in U.S. Pat. No. 5,565,186, issued Oct. 15, 1996.

MUTATION TABLE

| Pathogenic | Human | Sheep | Bovine |
|---|---|---|---|
| human mutations | Polymorphisms | Polymorphisms | Polymorphisms |
| 2 octarepeat insert | Codon 129 | Codon 171 | 5 or 6 octarepeats |
| | Met/Val | Arg/Glu | |
| 4 octarepeat insert | Codon 219 | Codon 136 | |
| | Glu/Lys | Ala/Val | |
| 5 octarepeat insert | | | |
| 6 octarepeat insert | | | |
| 7 octarepeat insert | | | |
| 8 octarepeat insert | | | |
| 9 octarepeat insert | | | |
| Codon 102 Pro-Leu | | | |
| Codon 105 Pro-Leu | | | |
| Codon 117 Ala-Val | | | |
| Codon 145 Stop | | | |
| Codon 178 Asp-Asn | | | |
| Codon 180 Val-Ile | | | |
| Codon 198 Phe-Ser | | | |
| Codon 200 Glu-Lys | | | |
| Codon 210 Val-Ile | | | |
| Codon 217 Asn-Arg | | | |
| Codon 232 Met-Ala | | | |

It should also be noted that such proteins have two different 3-dimensional conformations with the same amino acid sequence. One conformation is associated with disease characteristics and is generally insoluble whereas the other conformation is not associated with disease characteristics and is soluble. The methodology of the present invention is not limited to the diseases, proteins and strains listed

Cell Cultures

Another aspect of the invention involves the administration of a compound or compounds of the type described herein, into a cell culture, which cell culture is used for producing a drug such as a human drug. Particularly, human cells or Chinese hamster ovary (CHO) cells in culture can be used to produce genetically engineered drugs that are administered to humans. However, it is possible that the cells would themselves produce prions and thereby infect the product being produced with prions that, if administered to an animal, such as a human, would infect that animal. Thus, an aspect of the invention is combining a compound or compounds, as described herein with a cell culture, which cell culture is used in producing a drug and in particular used in producing a human drug. The compound is added in such an amount so as to inactivate prion infectivity and/or an amount so as to prevent prion formation and allow for the clearance of prions from the system such as the cell culture used in producing the drug of interest.

Chloropromazine and Quinacrine

In another aspect of the invention, a compound such as chloropromazine or quinacrine could be administered to a patient over a significant period of time when the patient is infected with a prion related disease such as CJD. This treatment is a non-prophylactic treatment, but rather a direct treatment of the patient such as a human in an attempt to cure what is now a 100% fatal disease. Chloropromazine is an anesthetic and would sedate the patient when administered in large amounts. If the patient were continually sedated over a significant period of time such as several days to several weeks (3 days to 3 weeks or more), the patient could then be revived and in a state where prions had been reduced or completely removed from the system of the patient.

Combination Therapy

It is a further aspect of the invention to use quinacrine and chlorpromazine in combination to treat diseases characterized by the presence of abnormal proteins. The use of the two compounds together has an unexpected synergistic effect. The combination may be in any ratio from 80:20 to 20:80 with a 50:50 mixture being preferred. The total dosing amounts is substantially the same as when using a single compound.

Compositions of PrP^{Sc} Antagonists

Inhibition of PrP^{Sc} formation or accumulation by tricyclic compounds with a middle ring side chain can inhibit amyloid fibril formation leading to cessation or stabilization of the neurodegenerative disease state. Use of appropriate compounds, such as, for example, phenothiazine or acridine derivative compounds that retain the ability to interfere with generation and/or accumulation of PrP^{Sc} protein are included within the scope of the present invention. Preferred compositions comprise phenothiazines and acridines, more preferred are promazine, chlorpromazine, quinacrine and acepromazine.

In one aspect, active agents are able to cross the blood-brain barrier of a human to produce a therapeutically efficacious concentration in cerebrospinal fluid and CNS tissues (e.g., cortical neurons). Other approaches to enhancing delivery of drugs, particularly across the blood-brain barrier, utilize osmotic pumps and/or pharmacologic-based procedures involving drug latentiation or the conversion of hydrophilic drugs into lipid-soluble drugs.

The present invention further comprises pharmaceutical compositions incorporating a compound of general structural formula (I or II or a combination thereof) in a pharmaceutically acceptable carrier or diluent. Such pharmaceutical compositions should contain a therapeutic or prophylactic amount of at least one compound identified by the method of the present invention. The pharmaceutically acceptable carrier can be any compatible, non-toxic substance suitable to deliver the compounds to an intended host. Sterile water, alcohol, fats, waxes, and inert solids may be used as the carrier.

Any suitable dosage can be given in the method of the invention. The type of compound and the carrier and the amount will vary widely depending on the species of the mammal, body weight, and disease being treated. The dosage administered will, of course, vary depending upon known factors, such as the pharmacodynamic characteristics of the particular agent and its mode and route of administration; the age, health and/or weight of the recipient; the nature and extent of the symptoms; the kind of concurrent treatment; the frequency of treatment; and the effect desired.

Administration can be oral or parenteral, with preferred administration routes including transdermal administration, subcutaneous injection, intravenous injection, intraperitoneal injection, intramuscular injection, intrasternal injection, intrathecal injection, intranasal and infusion techniques.

Pharmaceutically acceptable adjuvants, buffering agents, dispersing agents, and the like may also be incorporated into the pharmaceutical compositions. Preparation of pharmaceutical conditions incorporating active agents is well described in the medical and scientific literature. See, for example, Remington's Pharmaceutical Sciences, Mack Publishing Company, Easton, Pa., 16th Ed., 1982.

The pharmaceutical compositions just described are suitable for systemic administration to the host, including parenteral, topical, and oral administration, as well as intracranial administration.

A dosage unit can comprise a single compound or mixtures thereof with other compounds or other inhibiting compounds. The dosage unit can also comprise diluents, extenders, carriers and the like. The unit can be in solid or gel form such as pills, tablets, capsules and the like or in liquid form suitable for oral, rectal, topical, intravenous injection or parenteral administration or injection.

The compound derivatives are typically mixed with a pharmaceutically acceptable carrier. This carrier can be a solid or liquid and the type is generally chosen based on the type of administration being used. The active agent can be co-administered in the form of a tablet or capsule, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets can contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules.

Such liquid dosage forms can contain, for example, suitable solvents, preservatives, emulsifying agents, suspending agents, diluents, sweeteners, thickeners, and melting agents. Oral dosage forms optionally contain flavorants and coloring agents. Parenteral and intravenous forms would also include minerals and other materials to make them compatible with the type of injection or delivery system chosen. Thus, the present invention provides compositions for administration to a host, where the compositions comprise a pharmaceutically acceptable solution of the identified PrP^{Sc}-inhibitory compound in an acceptable carrier, as described above.

Compositions containing the present PrP^{Sc} inhibitors can be administered for prophylactic and/or therapeutic treatments of neurodegenerative disease. In therapeutic application, compositions are administered to a patient already affected by the particular neurodegenerative disease, in an amount sufficient to cure or at least partially arrest the condition and its complications. An amount adequate to accomplish this is defined as a “therapeutically effective dose” or “efficacious dose.”

The compounds of the present invention can be effective pharmaceuticals for therapy in pathogenic protein diseases, like prion related diseases in mammals. The PrP^{Sc} inhibiting concentrations of phenothiazine derivatives can be achieved in the brain by daily dosages commonly used in psychopharmacotherapy. See, Svendsen et al. (1988). For example, in patients treated with a daily oral dosage of 800 mg thioridazine, brain concentrations of more than 80 μM were achieved as measured in post mortem brain samples of different brain regions, and with 300 mg daily thioridazine, a concentration of 20 μM was achieved. Although chlorpromazine is believed to be slower at passing the blood brain barrier than other compounds, such as, thioridazine, concentrations achieved by the common dosage regimens are above the effective PrP^{Sc} inhibiting concentrations.

In the methods of the present invention, the compounds herein described in detail can form the active ingredient, and are typically administered in admixture with suitable pharmaceutical diluents, excipients, or carriers (collectively referred to herein as a pharmaceutically acceptable carrier or carrier materials) suitably selected with respect to the intended form of administration, that is, oral tablets, capsules, elixirs, syrups and the like, and consistent with conventional pharmaceutical practices.

For instance, for oral administration in the dosage unit form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like.

For oral administration in liquid dosage form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The derivatives can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Phenothiazine and acridine derivatives can also be coupled with soluble polymers as targetable drug carriers. Such polymers can include polyvinylpyrrolidone, pyran copolymer, polyhydroxylpropylmethacrylamide-phenol, polyhydroxyethylaspartamidephenol, or polyethyleneoxide-polylysine substituted with palmitoyl residues. Furthermore, the compounds of the present invention can be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The active ingredient can be administered orally in solid dosage forms, such as capsules, tablets, and powders, or in liquid dosage forms, such as elixirs, syrups, and suspensions. It can also be administered parentally, in sterile liquid dosage forms, including liposomes.

Gelatin capsules can contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient or a liposome, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company.

The present invention also includes pharmaceutical kits useful, for example, for the treatment of pathogenic protein diseases, which comprise one or more containers containing a pharmaceutical composition comprising a therapeutically effective amount of a compound of formula (I or II). Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit. In the present disclosure it should be understood that the specified materials and conditions are important in practicing the invention but that unspecified materials and conditions are not excluded so long as they do not prevent the benefits of the invention from being realized.

The invention also provides the use of a PrP^{Sc} inhibitor to slow, arrest, or reverse the development of a neurodegenerative disease in a human patient; an efficacious amount of the PrP^{Sc} inhibitor is administered to the patient to inhibit progression of the disease.

Livestock Feed

An important composition of the invention is a combination of a compound, which prevents prion formation and/or aids in the clearance of prion from a mammal combined with a livestock feed. In particular a compound of the invention is combined with a livestock feed derived from an animal source, such as meat or bone meal and more particularly animal material that includes ground material from the central nervous system from another animal. Such feed may be infected with prions. However, the compounds of the invention can be combined with plant derived materials used in livestock feed in order to prevent or treat prion infections in the animal eating the feed. More particularly, the compounds of the present invention can be added to animal feed or feedstuffs used to feed any type of mammal and particularly for livestock. The feedstuff compositions disclosed herein are intended to provide nutritional requirements of a variety of animals, including cattle, poultry, swine, sheep, goats, other monogastric or ruminant livestock. The composition generally varies according to the type of animals to which the feedstuff will be given. Examples of various animal feedstuff components can be found in U.S. Pat. No. 6,207,217, U.S. Pat. No. 6,203,843, U.S. Pat. No. 5,786,007, U.S. Pat. No. 4,225,621, U.S. Pat. No. 4,161,543 and U.S. Pat. No. 4,062, 988.

Generally, when the term “feedstuff” is used with respect to the present invention, the term comprises all types of plant and animal components. Specifically, “feedstuff” organic components such as proteins, crude fiber, acid detergent fiber, neutral detergent fiber, vitamins and minerals. Typical compositions of feedstuffs for livestock include, but are not limited to, the following components: alfalfas, ammonium sulfate, barleys, beet pulps, blood meal, bluestem grass, brewers grains and yeast, brome grass, calcium carbonate, canary grass, carrot pulp, roots and tops, cattle manure, cheatgrass, clovers, coffee grounds, corn and corn plants, cottonseed, defluorinated phosphate, diammonium phosphate, dicalcium phosphate, distillers grain barley, distillers grain corn, feathermeal hydrolyzed, garbage (municipal), grain screenings and grain dust, grape pomace, grass silage, hominy feed, hop leaves, vines and spent hops, limestone, linseed meal, all types of hay including meadow hay, meat and bone meal (MBM), milo grain, mint slug silage, molasses beet, cane, citrus and wood, monoammonium phosphate, mono-dicalcium phosphate, navy beans, all types of oats including oat hay, oat silage, oat straw, oat grain, groats, oat meal, oat mill byproducts and oat hull, orange pulp, orchard grass, pea vines, peanut hulls, skins and meal, potato vine, potatoes and potato waste, poultry fat and poultry litter and manure, prairie hay, rapemeal solvent, rye straw and grain, safflower meal, sagebrush, sorghum stover and silage, soybeans and soybean hull, sudangrass hay and silage, sunflower meal and hulls, timothy hay and silage, tomatoes, triticale silage, urea, wheat bran, wheat grass, wheat grain, wheat shorts and wheat straw. Further, the above feedstuff components are set forth above serve merely as examples and are not intended to be comprehensive or limiting. As such, suitable feedstuffs for the present invention may comprise additional components not provided in the list above.

Of the above listed feed components, meat and bone meal (MBM) stands out as one the richest sources of energy and minerals. Typically, the crude protein content of MBM is about 50%. See, Hamilton, C. R., “Meat and Bone Meal,” Esteem Products. Vol. 1(1). MBM is thus one of the most efficient feed components. MBM is produced as a by-product from the removal of fat from animal tissues through rendering. The rendering process produces a finely ground, dry residue of animal by-products pressure cooked and stabilized by high temperature steam in closed tanks. The fat can be skimmed off and the solid residue is pressed to remove as much of the fat and water as possible. As defined and regulated by the Association of American Feed Control Officials (AAFCO), MBM is the rendered product from mammal tissues, including bone, exclusive of any added blood, hair, hoof, horn, hide trimmings, manure, stomach and rumen contents, except in such amounts as may occur unavoidably in good processing practices. As such, neuronal tissues are included in MBM products. See, “The BSE Inquiry” § 9.15 at
http://www.bse.org.uk/report/volume7/chapteh2.htm.

The invention comprises feedstuff as defined, in combination with a compound that inhibits prion formation. A compound of the invention such as a compound having the structural formula (I) and/or (II) is added to feedstuff and fed to an animal and in particular to domesticated livestock farm animals such as cows, pigs, sheep, goats, horses, chickens, etc. The active compound is added in an amount sufficient to “treat” the animal. The amount will vary based on factors such as the type of animal and its size. In general, dosing is such that the animal will receive about 10 mg to about 10,000 mg/day/kg of weight of the animal.

Methods For Identifying PrP^{Sc} Antagonists

Until the development of the present invention, identification of active agents which inhibit the development of prion related diseases was governed by simple brute force, screening of all possible chemical structures in a suitable cellular and/or animal model of apoptotic neurotoxicity. As such, the complexity and structural potential of chemistry makes a thorough search of all of the chemical structural space impossible, even if facile synthetic methods were available for all potential compounds. Because an exhaustive search of chemical space is not possible, it is exceedingly important to identify properties of likely inhibitors of neurodegenerative processes involved in prion related diseases. The present invention fulfills this goal.

In order to expedite the screening of compound libraries and to increase the probability of obtaining active agents which inhibit PrP^{Sc} and neurodegeneration, it is desirable to preselect compounds which are known or suspected inhibitors of PrP^{Sc} (based on structural homology to the class of compounds identified herein), and preferably are selective inhibitors of PrP^{Sc}.

PrP^{Sc} inhibitors are typically identified by initially employing a PrP^{Sc} inhibition assay, which may comprise using scrapie-infected neuroblastoma cells (ScN2a). For example, a primary PrP^{Sc} assay can be performed according to the PrP^{Sc} inhibition assay of the present invention as shown in the Examples. Agents that are found to inhibit PrP^{Sc} activity in the assay are then selected for subsequent testing in a secondary assay, such as being administered to transgenic mice.

Other suitable assays for measuring the capacity of an agent to inhibit PrP^{Sc} will be apparent to those in the art in view of the specification. The primary PrP^{Sc} assays can also be multiplexed, such that agents, which are positively identified in one primary assay, are verified as bona fide PrP^{Sc} inhibitors in another type of primary assay.

Agents selected in the primary assay(s) as PrP^{Sc} inhibitors are evaluated for their capacity to inhibit PrP^{Sc} or neuronal degeneration or the like. Secondary assays can measure the ability of a selected agent to inhibit neurodegeneration in neurodegenerative disease models. Typically, a secondary assay is performed using a primary rat or human cortical or hippocampal neuron culture and/or a rat or human cortical or hippocampal astrocyte/microglia culture, as described herein; alternatively, a neuronal cell line can be employed, typically with (1) primary glial cells and/or a glial cell line, and/or (2) primary astrocytes and/or an astrocytic cell line (astrocytoma cells). However, other suitable neurodegeneration models can be employed, such as transgenic mice expressing an amyloidogenic polypeptide and exhibiting neuropathology (e.g., a PrP transgenic mouse). A plurality of secondary assays may also be multiplexed, so that for example agents which score positive as in a neuronal cell culture neurodegeneration inhibition assay can be tested in a mammalian model of neurodegenerative disease (e.g., a transgenic mouse PrP model), and vice versa.

Thus, a primary screening assay to identify PrP^{Sc} inhibitors can be performed prior to a secondary screening assay. An advantage of this approach is that it substantially reduces the number of chemical structures that need to be searched to identify neurodegeneration inhibitors.

The following examples are provided for illustration and are not intended to limit the invention to the specific example provided.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

In Vitro Experiments Of Permanently Scrapie-Infected Neuroblastoma Cells (Scn2a)

The formation and accumulation of PrP^{Sc} in mammalian cells can be inhibited by the administration of compounds with a tricyclic ring structure and a mid-ring side chain, such as, for example, phenothiazine derivatives and quinacrine.

Scrapie-infected mouse neuroblastoma cells were used as a model to study prion protein (PrP) formation and accumulation. Identically seeded N2a scrapie-infected neuroblastoma cells were infected with an RML strain of mouse adapted scrapie prions and subclones. A confluent 10 cm² dish was split and cells were pipetted into a 60 mm² dish of 4 ml MEM containing 10% FCS, penicillin-streptomycin and nonessential amino acids. The medium was exchanged every two days, together with the test compound. Cells were lysed (lysis buffer, 10 mM Tris pH 8.0, 150 mM NaCl, 0.5% NP40, 0.5% desoxycholate) on the seventh day having achieved an 80% confluency.

In brief, cell lysates were digested with proteinase K at 20 μg/ml for 30 minutes at 37° C. The reaction was stopped with 2 mM PMSF and lysates were centrifuged for 45 minutes at 100,000×g. Pellets were resuspended in sample buffer and analyzed by sodium dodecyl sulfate (SDS)-polyacrylamide gel electrophoresis (PAGE) and immunoblotting according to standard techniques. Immunoblots were incubated with recombinant Fab antibody D13, a secondary horseradish peroxoidase labeled antibody and developed with an ECL system (Amersham, USA). Densitometry was performed with NIH image software.

### Example 2

Differential Accumulation of Proteinase K Resistant PrP^{Sc} in ScN2a Cells Treated With, Two Optical Isomers of Quinacrine.

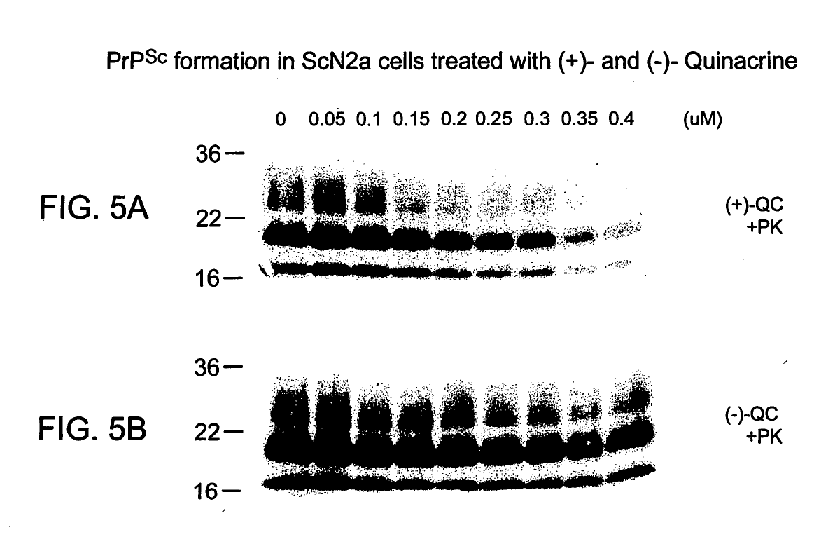

This experimental involves a comparison of two optical isomers of quinacrine specifically the dextrorotary and laevorotary isomers. The comparison clearly shows that the dextrorotary isomer (100% D) shown in FIG. 5A as the (+) optical isomer has substantially greater activity as compared to the (L) optical isomer (100% L). Within the example ScN2a cells are cultivated in MEM with 10% fetal calf serum and GlutaMax. For treatments, ScN2a cells were plated at about 2-3% confluency. Then, the indicated concentration shown in FIGS. 5A and 5B of 0-0.4 micromolar of optical (D) and (L) quinacrine isomers were added to the cell cultures. The cell media including quinacrine were changed every alternate day and treatment lasted for six days. Western blots of proteinase-K digested ScN2a cell lysates were incubated with (+)-and(−)-quinacrine and the results of such are shown in FIGS. 5A and 5B respectively. Molecular weight size markers (Kda) are shown in the left hand side of FIG. 5A for (+)-dextrorotary and in FIG. 5B for (−)-laevorotary, quinacrine, +PK; proteinase K-digested.

The foregoing detailed description has exemplified the discoveries with reference to certain particular compounds and their role in treating neurodegenerative diseases. Other discoveries also form a part of the present invention. Thus, the scope of the present invention can be interpreted with reference to the appended claims.

1. A method of treating disease resulting from malformed proteins from a mammal comprising:
administering to said mammal a compound selected from the group consisting of quinacrine and chlorpromazine; wherein said compound is characterized by clearing malformed proteins and by an ability to cross a blood brain barrier of said mammal.

2. The method of claim 1, wherein said mammal is selected from the group consisting of a cow, pig, sheep and goat.

3. The method of claim 1, wherein the compound is quinacrine and the quinacrine consists of the dextrorotary optical isomer.

4. A method of treating a human having a malformed protein, comprising:
administering to said human a compound comprising a pharmaceutically acceptable carrier, and a compound selected from the group consisting of quinacrine and chlorpromazine,
wherein said administration is in a therapeutically effective amount.

5. The method of claim 4, wherein the administration is an oral administration.

6. The method of claim 4, wherein the malformed protein and its associated disease is selected from the group consisting of:

| Disease | Insoluble Proteins |
|---|---|
| Alzheimer's Disease | APP, Aβ peptide, |
| | α1-antichymotrypsin, |
| | tan, non-Aβ component |
| Prion diseases, | |
| Creutzfeld Jakob disease, | |
| scrapie and bovine | |
| spongeform | |
| Encephalopathy | PrP^{Sc} |
| ALS | SOD and neurofilament |
| Pick's disease | Pick body |
| Parkinson's disease | Lewy body |
| Diabetes Type 1 | Amylin |
| Multiple myeloma— | IgGL-chain |
| plasma cell dyscrasias | |
| Familial amyloidotic | Transthyretin |
| polyneuropathy | |
| Medullary carcinoma of | Procalcitonin |
| thyroid | |
| Chronic renal failure | β₂—microglobulin |
| Congestive heart failure | Atrial natriuretic factor |
| Senile cardiac and | Transthyretin |
| systemic amyloidosis | |
| Chronic inflammation | Serum amyloid A |
| Atherosclerosis | ApoA1 |
| Familial amyloidosis | Gelsolin. |

7. The method of claim 4, wherein the disease and its associated malformed prion is selected from the group consisting of

| | |
|---|---|
| Alzheimer's Disease | APP, Aβ peptide, α1- |
| | antichymotrypsin, tan, non- |
| | Aβ component |
| Prion diseases, Creutzfeld | |
| Jakob disease, scrapie and | |
| bovine spongeform | |
| Encephalopathy | PrP^{Sc} |
| Parkinson's disease | Lewy body |
| Diabetes Type 1 | Amylin |
| Familial amyloidotic | Transthyretin. |
| polyneuropathy | |

8. The method according to claim 5, wherein the oral administration step is in an amount of about 100 mg to 10,000 mg/day/75 kg of body weight.

9. The method of claim 4, wherein the administration step comprises administration by injection.

10. The method of claim 4, wherein the administration step comprises a technique selected from the group consisting of transdermal administration, subcutaneous injection, intravenous injection, intraperitoneal injection, intramuscular injection, intrasternal injection, intrathecal injection, intranasal, and infusion techniques.

11. The method as claimed in claim 4, wherein the compound is quinacrine and the quinacrine is 100% dextrorotary quinacrine.

12. A method of treating a disease resulting from malformed proteins comprising:
administering to said mammal a pharmacologically effective amount of a combination of quinacrine and chlorpromazine.

13. The method as claimed in claim 12, wherein the quinacrine consists of dextrorotary quinacrine.

14. The method of claim 12, wherein the mammal is suffering from Creutzfeldt-Jakob disease.

15. The method of claim 12, wherein the mammal is suffering from a disease selected from the group consisting of scrapie, transmissible spongioform encephalopathy (TSE), Alzheimer's disease, Parkinson's disease, amyotrophic lateral sclerosis, multiple sclerosis, autism, schizophrenia, bipolar disorders, fronto-temporal dementia, Pick's disease, progressive supranuclear palsy, diffuse Lewy body disease, systemic lupus erythematosus, rheumatoid arthritis, Huntington's disease, spinocerebellar ataxias, diabetes mellitus, Types I and II, Crohn's disease, ulcerative colitis, systemic amyloidosis, primary amyloidosis, polyneuropathy and AIDS.

16. A composition for treating livestock with malformed proteins comprising:
livestock feed; and
quinacrine and chlorpromazine.

17. A composition for treating livestock with malformed proteins comprising:
livestock feed; and
a compound selected from the group consisting of quinacrine and chlorpromazine.

18. A method for clearing malformed proteins from livestock, said method comprising:
a. administering a pharmaceutically effective amount of the composition of claim 16; and
b. continuously providing said livestock feed to livestock.

19. A method for clearing malformed proteins from livestock, said method comprising:
a. administering a pharmaceutically effective amount of the composition of claim 17; and
b. continuously providing said livestock feed to livestock.

20. The composition of claim 16, wherein the quinacrine is 60% dextrorotary and 40% laevorotary.

21. A composition, comprising:
livestock feed; and
quinacrine.

22. The composition of claim 21, wherein the quinacrine is 60% or more dextrorotary quinacrine with the remainder being laevorotary quinacrine.

23. The composition of claim 16, wherein the quinacrine is 70% or more dextrorotary quinacrine with the remainder being laevorotary quinacrine.

24. The composition of claim 16, wherein the quinacrine is 80% or more dextrorotary quinacrine with the remainder being laevorotary quinacrine.

25. The composition of claim 16, wherein the quinacrine is 90% or more dextrorotary quinacrine with the remainder being laevorotary quinacrine.

26. The composition of claim 16, wherein the quinacrine is 100% dextrorotary quinacrine.

27. A composition for treating disease resulting from malformed proteins from a mammal comprising:
a compound selected from the group consisting of quinacrine and chlorpromazine; wherein said compound is characterized by clearing malformed proteins and by an ability to cross a blood brain barrier of said mammal.

28. The composition of claim 27, wherein the compound is quinacrine or a compound where the quinacrine consists of the dextrorotary optical isomer.

29. A composition for treating a human having a malformed protein, comprising:
a compound comprising a pharmaceutically acceptable carrier, and a compound selected from the group consisting of quinacrine and chlorpromazine,
wherein the compound is present in the composition is in a therapeutically effective amount.

30. A method of treating a disease resulting from malformed proteins, comprising:
administering to a patient a compound having the following general structural formula I: 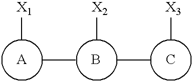
wherein each A, B and C are each independently a cyclic moiety and is optionally, substituted with X₁, X₂ and X₃ which are each independently hydrocarbyl.
